(19) United States
(12) Patent Application Publication
(10) Pub. No.: US 2026/0232765 A1
(43) Pub. Date: Aug. 13, 2026

(54) ACIDIC FIBROBLAST GROWTH FACTOR EYE DROP PREPARATION

(71) Applicant: SHANGHAI TENRY PHARMACEUTICAL CO., LTD., Shanghai (CN)

(72) Inventors: Dongjun FU, Shanghai (CN); Guoxing ZHU, Shanghai (CN); Fang TANG, Shanghai (CN); Jiaxin HE, Shanghai (CN); Kai WANG, Shanghai (CN); Zhengwu LI, Shanghai (CN); Lufan GUAN, Shanghai (CN)

(21) Appl. No.: 19/155,026

(22) PCT Filed: Jan. 3, 2024
(86) PCT No.: PCT/CN2024/070255

§ 371 Date: Aug. 8, 2025

(30) Foreign Application Priority Data
Apr. 21, 2023 (CN) 202310431075.4

(51) Int. Cl. A61K 38/18; A61K 9/00; A61K 47/02; A61K 47/06; A61K 47/20; A61K 47/22; A61K 47/26; A61K 47/32; A61K 47/36; A61K 47/38

(52) U.S. Cl. CPC A61K 38/1825; A61K 9/0048; A61K 47/02; A61K 47/06; A61K 47/20; A61K 47/22; A61K 47/26; A61K 47/32; A61K 47/36; A61K 47/38

(57) ABSTRACT

The present invention relates to a recombinant human acidic fibroblast growth factor preparation, and particularly relates to an acidic fibroblast growth factor eye drop preparation. In the eye drop preparation, a combination selected from histidine, Tween 80 and heparin sodium is used as a protein protective agent, without using human albumin as the protein protective agent, thereby reducing the costs, effectively improving the stability of the product, and maintaining high bioactivity for 9 months under the conditions of (25±2)° C. and relative humidity (60±5)%.

### TECHNICAL FIELD

The present invention relates to the field of biomedicine, and particularly to a stable acidic fibroblast growth factor preparation.

### BACKGROUND ART

Fibroblast growth factors (FGFs) are multifunctional and powerful cytokines that play an important role in promoting fibroblast metabolism and collagen formation. Fibroblast growth factors can promote the growth and reproduction of skin tissue. They regulate the division, reproduction, and growth differentiation of skin epithelial, endothelial and stromal cells, promote cell metabolism and enhance oxidation by binding to specific receptors on the cell surface. They can promote the rapid growth and reproduction of cells related to skin damage, and regulate the synthesis, secretion, and decomposition of the intercellular matrix. They can promote the regeneration of stratum corneum cells, accelerate the repair of the stratum corneum and matrix layer of the skin, and promote the growth of human skin cells. They can enhance the synthesis of protein and cell metabolism of skin cells, and have the effects of delaying skin cell aging and promoting the repair and growth of epidermal cells.

A fibroblast growth factor eye drop has an effect of promoting the repair and regeneration of cells derived from the mesoderm and ectoderm. The results of animal experiments show that the present product promotes the regeneration of corneal epithelium and the repair of corneal stroma and endothelium in rabbits with alkali burns; commercially available products include acidic fibroblast growth factor (aFGF or FGF-1) lyophilized powder and alkaline fibroblast growth factor (bFGF or FGF-2) eye drop. aFGF lyophilized powder needs to be stored and transported at 2-8° C. in a light-protected environment, and it has a shelf life of about 24 months. When used, the solvent provided in the package is poured into the bottle containing rhaFGF lyophilized powder. bFGF eye drop may be used directly, but must be stored at 2-8° C. and it has a shelf life of approximately 24 months. Once placed at room temperature, it should be used as soon as possible. Therefore, it is of great significance to study the stabilizer suitable for aFGF, obtain a stable aFGF pharmaceutical composition, and improve the compliance and convenience of patients; there is an urgent need in the art to provide a liquid preparation that can maintain the long-term stability of acidic fibroblast growth factor.

### SUMMARY OF THE INVENTION

In view of the deficiencies in the prior art, the present invention provides a stable acidic fibroblast growth factor preparation and preparation method and application thereof. The components of the preparation are studied, in particular, different combinations of stabilizers are selected and the proportioning of each stabilizer is compared. A stable preparation containing acidic fibroblast growth factor is obtained through repeated experimental studies.

The present invention provides a human acidic fibroblast growth factor eye drop, which comprises a protein protective agent, a humectant, a buffer salt, and an osmotic pressure regulator, wherein the protein protective agent is selected from a combination of histidine, Tween 80, and heparin sodium.

The present invention provides a recombinant human acidic fibroblast growth factor eye drop, wherein each 100 ml of the eye drop contains 0.01-0.2 g of histidine, 0.01-0.2 g of Tween 80 and 0.005-0.1 g of heparin sodium; preferably contains 0.1-0.2 g of histidine, 0.03-0.1 g of Tween 80 and 0.01-0.05 g of heparin sodium.

The present invention provides a recombinant human acidic fibroblast growth factor eye drop, wherein each 100 ml of the eye drop contains 1 to 3 mg of the human acidic fibroblast growth factor, preferably contains 2 mg of the human acidic fibroblast growth factor.

The present invention provides a recombinant human acidic fibroblast growth factor eye drop, wherein the humectant is selected from one or more of sodium hyaluronate, chondroitin sulfate, polyvinyl alcohol, polyethylene glycol, glycerol, hypromellose, dextran, and carboxymethyl cellulose, and each 100 ml of the eye drop contains 0.05 to 1.5 g of the humectant, and preferably, each 100 ml of the eye drop contains 0.1 g of the humectant.

The present invention provides a recombinant human acidic fibroblast growth factor eye drop, wherein the buffer salt is selected from the group consisting of phosphate buffer system, histidine salt buffer system, acetate buffer system, citrate buffer system, Tris buffer system, and HEPES buffer system, and the preferred pH value of the buffer salt is 6.0 to 7.0, the concentration is 0.01 to 0.05 mol/L, and the preferred concentration is 0.02 mol/L.

The present invention provides a recombinant human acidic fibroblast growth factor eye drop, wherein the osmotic pressure regulator is selected from one or more of sodium chloride, boric acid and glucose, and preferably, each 100 ml of the eye drop contains 0.5 to 0.9 g of the osmotic pressure regulator.

The present invention provides a recombinant human acidic fibroblast growth factor eye drop, wherein the eye drop further contains an antibacterial agent.

The present invention provides a recombinant human acidic fibroblast growth factor eye drop, wherein the antibacterial agent is selected from one or more of ethylparaben, benzalkonium chloride, benzalkonium bromide, polyquaternium salt, phenylmercuric nitrate, thimerosal, benzalkonium chloride, trichlorotert butanol, hydroxyphenyl esters, and sorbitol, and preferably, each 100 ml of the eye drop contains 0.01 to 0.1 g of the antibacterial agent.

The present invention provides a recombinant human acidic fibroblast growth factor eye drop, which further comprises an antioxidant, and the antioxidant is selected from one or more of sodium thiosulfate, sodium sulfite, vitamin E, sodium ascorbate, and methionine, and preferably, each 100 ml of the eye drop contains 0.01 to 0.1 g of the antioxidant.

The present invention provides a recombinant human acidic fibroblast growth factor eye drop, which further contains a thickener; preferably, the thickener is selected from one or more of polyvinyl alcohol, carboxymethyl cellulose, methyl cellulose, sodium carboxymethyl cellulose, sodium polyacrylate, and polyvinyl pyrrolidone; and preferably, each 100 ml of the eye drop contains 0.5 to 2 g of the thickener.

The present invention has the following beneficial effects:

The eye drop of the present invention uses a combination of histidine, Tween 80 and heparin sodium as a protein protective agent, which effectively improves the stability of the product and still maintains a high biological activity for 9 months under the conditions of (25±2)° C. and relative humidity (60±5) %.

The combined protein protective agent used in the present invention does not need to use expensive human albumin, and effectively reduces the production costs while ensuring product quality.

Through screening, the present inventors have found a variety of preferred combinations of antibacterial agents and antioxidants, which facilitate in synergistically increasing the stability of the product.

### DETAILED DESCRIPTION OF EXAMPLES

In order to facilitate a better understanding of the present invention, the following examples are used to illustrate the present invention. These examples belong to the protection scope of the present invention, but do not intend to limit the protection scope of the present invention.

Example 1: The present example provides a recombinant human acidic fibroblast growth factor preparation I, the formulation and preparation method of which are as follows:

| | | |
|---|---|---|
| Human acidic fibroblast growth factor | 2 | mg |
| Sodium hyaluronate | 0.1 | g |
| Histidine | 0.15 | g |
| Tween 80 | 0.05 | g |
| Heparin sodium | 0.02 | g |
| Sodium chloride | 0.45 | g |
| Trichlorotert butanol | 0.05 | g |
| Methionine | 0.05 | g |
| Polyvinyl alcohol | 1 | g |

The preparation of 100 ml of the eye drop: in accordance with the above-described proportions, place the humectant (sodium hyaluronate), protein protective agent (histidine, Tween 80, and heparin sodium), thickener (polyvinyl alcohol), preservative (trichlorotert butanol), and antioxidant (methionine) into 80 ml of phosphate buffer (0.02 mol/L); add 0.9 g of sodium chloride, and following complete dissolution, add the human acidic fibroblast growth factor. After thorough mixing, adjust the volume to the required volume with phosphate buffer (pH 6.5, 0.02 mol/L), sterilize the solution by filtration through a 0.22 μm microporous membrane, and dispense the filtrate into plastic eye drop bottles to prepare the finished eye drops of the present invention.

Example 2: The preparation method of the present example is the same as that of Example 1, and the specific formulation is as follows:

| | | |
|---|---|---|
| Human acidic fibroblast growth factor | 2 | mg |
| Sodium hyaluronate | 0.1 | g |
| Histidine | 0.15 | g |
| Tween 80 | 0.05 | g |
| Heparin sodium | 0.05 | g |
| Sodium chloride | 0.45 | g |
| Trichlorotert butanol | 0.05 | g |
| Sodium thiosulfate | 0.05 | g |
| Polyvinyl alcohol | 1 | g |

Example 3: The preparation method of the present example is the same as that of Example 1, and the specific formulation is as follows:

| | | |
|---|---|---|
| Human acidic fibroblast growth factor | 2 | mg |
| Sodium hyaluronate | 0.1 | g |
| Histidine | 0.15 | g |
| Tween 80 | 0.05 | g |
| Heparin sodium | 0.05 | g |
| Sodium chloride | 0.45 | g |
| Methyl p-Hydroxybenzoate | 0.05 | g |
| Methionine | 0.05 | g |
| Polyvinyl alcohol | 1 | g |

Example 4: The preparation method of the present example is the same as that of Example 1, and the specific formulation is as follows:

| | | |
|---|---|---|
| Human acidic fibroblast growth factor | 2 | mg |
| Sodium hyaluronate | 0.1 | g |
| Histidine | 0.15 | g |
| Tween 80 | 0.05 | g |
| Heparin sodium | 0.05 | g |
| Sodium chloride | 0.45 | g |
| Sodium benzoate | 0.05 | g |
| Methionine | 0.05 | g |
| Polyvinyl alcohol | 1 | g |

Comparative Example 1: The preparation method of the present comparative example refers to Example 1, and the specific formulation is as follows:

| | | |
|---|---|---|
| Human acidic fibroblast growth factor | 2 | mg |
| Sodium hyaluronate | 0.1 | g |
| Tween 80 | 0.05 | g |
| Heparin sodium | 0.05 | g |
| Sodium chloride | 0.45 | g |
| Trichlorotert butanol | 0.05 | g |
| Methionine | 0.05 | g |
| Polyvinyl alcohol | 1 | g |

Comparative Example 2: The preparation method of the present comparative example refers to Example 1, and the specific formulation is as follows:

| | | |
|---|---|---|
| Human acidic fibroblast growth factor | 2 | mg |
| Sodium hyaluronate | 0.1 | g |
| Histidine | 0.15 | g |
| Heparin sodium | 0.05 | g |
| Sodium chloride | 0.45 | g |
| Trichlorotert butanol | 0.05 | g |
| Methionine | 0.05 | g |
| Polyvinyl alcohol | 1 | g |

Comparative Example 3: The preparation method of the present comparative example refers to Example 1, and the specific formulation is as follows:

| | | |
|---|---|---|
| Human acidic fibroblast growth factor | 2 | mg |
| Sodium hyaluronate | 0.1 | g |
| Histidine | 0.15 | g |
| Tween 80 | 0.05 | g |
| Sodium chloride | 0.45 | g |
| Trichlorotert butanol | 0.05 | g |
| Methionine | 0.05 | g |
| Polyvinyl alcohol | 1 | g |

Comparative Example 4: The preparation method of the present comparative example refers to Example 1, and the specific formulation is as follows:

| | | |
|---|---|---|
| Human acidic fibroblast growth factor | 2 | mg |
| Sodium hyaluronate | 0.1 | g |
| Heparin sodium | 0.05 | g |
| Human albumin | 0.1 | g |
| Sodium chloride | 0.45 | g |
| Methionine | 0.05 | g |
| Polyvinyl alcohol | 1 | g |

Comparative Example 5: The preparation method of the present comparative example refers to Example 1, and the specific formulation is as follows:

| | | |
|---|---|---|
| Human acidic fibroblast growth factor | 2 | mg |
| Sodium hyaluronate | 0.1 | g |
| Histidine | 0.15 | g |
| Human albumin | 0.1 | g |
| Sodium chloride | 0.45 | g |
| Trichlorotert butanol | 0.05 | g |
| Polyvinyl alcohol | 1 | g |

### Effect Example: Accelerated Stability Test

The eye drops prepared in Examples 1-4 and Comparative Examples 1-4 were placed under conditions of (25±2° C.) and relative humidity (60±5) % for 9 months, and then an activity test was performed. The activity test method was as follows:
(1) Preparation of standard solution: take the recombinant human acidic fibroblast growth factor standard, reconstitute it according to the instructions under sterile conditions, and dilute it to contain 40 IU per 1 ml. In a 96-well cell culture plate, perform a 4-fold serial dilution, with 8 dilution levels, and 2 wells for each dilution level.
(2) Preparation of test sample solution: dilution method and treatment method are the same as those of standard samples.
(3) The above samples were immediately subjected to the test in step (4) below.
(4) Activity assay: Balb/c 3T3 cell line was cultured in complete culture medium at 37° C. and 5% CO₂, and the cell concentration was controlled at 1.0×10⁵ to 5.0×10⁵ cells/ml. The cells were used for biological activity assay 24 to 36 hours after subculture. The culture medium in the culture flask was discarded, the cells were digested and collected, and a cell suspension of 5.0×10⁴ to 1.3×10⁵ cells/ml was prepared with complete culture medium, and inoculated into a 96-well cell culture plate with 100 μL per well. Incubation was performed at 37° C. and 5% CO₂ for 24 h. Then, the culture medium was replaced with maintenance medium and cultured at 37° C. and 5% CO₂ for 28 to 32 hours. The maintenance solution was discarded from the prepared cell culture plate, and the standard solution and the test sample were added with 100 μL per well. Incubation was performed at 37° C. and 5% CO₂ for 64 to 72 hours. At the end of the culture, 20 μL of MTT solution was added to each well and cultured at 37° C. and 5% CO₂ for 5 to 6 hours. 100 μL of lysis solution was added to each well of the cell culture plate, which was mixed well and put into the microplate reader, and 630 nm was used as the reference wavelength. The absorbance at a wavelength of 570 nm was measured, and the measurement results were recorded.

Result calculation: The test data were processed by computer program or Linear regression calculation method, and the results were calculated as follows:

Biological activity of the test sample (IU/ml)=Pr×Ds×Es/(Dr×Er)

Relative activity (%)=biological activity of the test sample (after accelerated test)/biological activity of the test sample (at the stability baseline)

Where Pr is the biological activity of the standard, in IU/ml; Ds is the pre-dilution factor of the test sample; Dr is the pre-dilution factor of the standard; Es is the dilution factor of the test sample equivalent to the half-effective dose of the standard; Er is the dilution factor of the half-effective dose of the standard; the relative activity (%) test data after the accelerated test is shown in Table 1:

| | | | | | Comparative | Comparative | Comparative | Comparative | Comparative |
|---|---|---|---|---|---|---|---|---|---|
| | Example 1 | Example 2 | Example 3 | Example 4 | Example 1 | Example 2 | Example 3 | Example 4 | Example 5 |
| relative | 92.5 | 98.3 | 93.1 | 89.3 | 58.6 | 53.7 | 46.1 | 61.1 | 63.6 |
| activity | | | | | | | | | |
| (%) | | | | | | | | | |

The above data show that the combination of histidine, Tween 80 and heparin sodium as a combination of protein protective agent is beneficial to improve the stability of human acidic fibroblast growth factor under accelerated test conditions; as for the effect of antioxidants and preservatives on stability, the combination of trichlorotert butanol and methionine in Example 1 is the best embodiment, and the effects of other preservatives and antioxidants are slightly worse.

The above contents cannot be used to determine that the specific implementation of the present invention is limited to these descriptions. Those skilled in the art to which the present invention belongs can make several simple deductions or substitutions without departing from the concept of the present invention, which should be regarded as belonging to the protection scope of the patent of the present invention determined by the submitted claims.

1. A recombinant human acidic fibroblast growth factor eye drop, the eye drop comprising a protein protective agent, a humectant, a phosphate buffer system, and an osmotic pressure regulator, an antibacterial agent and an antioxidant, wherein the protein protective agent is selected from the combination of histidine, Tween 80, and heparin sodium, and each 100 ml of the eye drop contains 0.1-0.2 g of histidine, 0.03-0.1 g of Tween 80 and 0.01-0.05 g of heparin sodium;
wherein the antibacterial agent and the antioxidant are
trichlorotert butanol and methionine, respectively,
trichlorotert butanol and sodium thiosulfate, respectively, or
methyl p-hydroxybenzoate and methionine, respectively;
wherein each 100 ml of the eye drop contains 0.01 to 0.1 g of the antibacterial agent, and each 100 ml of the eye drop contains 0.01 to 0.1 g of the antioxidant; and
wherein the phosphate buffer system has a pH value of 6.0 to 7.0; the osmotic pressure regulator is selected from sodium chloride, and each 100 ml of the eye drop contains 0.3 to 0.9 g of the osmotic pressure regulator.

2. (canceled)

3. The recombinant human acidic fibroblast growth factor eye drop according to claim 1, wherein each 100 ml of the eye drop contains 1 to 3 mg of a human acidic fibroblast growth factor.

4. The recombinant human acidic fibroblast growth factor eye drop according to claim 1, wherein the humectant is selected from one or more of sodium hyaluronate, chondroitin sulfate, polyvinyl alcohol, polyethylene glycol, glycerol, hypromellose, dextran, and carboxymethyl cellulose, and each 100 ml of the eye drop contains 0.01 to 1.5 g of the humectant.

5. The recombinant human acidic fibroblast growth factor eye drop according to claim 1, wherein the phosphate buffer system has a pH value of 6.0 to 7.0, and a concentration of 0.01 to 0.05 mol/L.

6-10. (canceled)

11. The recombinant human acidic fibroblast growth factor eye drop according to claim 1, wherein the eye drop further contains a thickener; and the thickener is selected from one or more of polyvinyl alcohol, carboxymethyl cellulose, methyl cellulose, sodium carboxymethyl cellulose, sodium polyacrylate, and polyvinyl pyrrolidone; and each 100 ml of the eye drop contains 0.5 to 2 g of the thickener.

12. The recombinant human acidic fibroblast growth factor eye drop according to claim 1, wherein each 100 ml of the eye drop contains 2 mg of a human acidic fibroblast growth factor.

13. The recombinant human acidic fibroblast growth factor eye drop according to claim 4, wherein, each 100 ml of the eye drop contains 0.1 g of the humectant.

14. The recombinant human acidic fibroblast growth factor eye drop according to claim 1, wherein the phosphate buffer system has a concentration of 0.02 mol/L.
